(19) United States
(12) Patent Application Publication
(10) Pub. No.: US 2005/0273139 A1
(43) Pub. Date: Dec. 8, 2005

(54) Device for clamping tissue

(76) Inventors: Norbert Krauss, Nuernberg (DE); Rainer Kuth, Herzogenaurach (DE); Johannes Reinschke, Nuernberg (DE)

Correspondence Address: HARNESS, DICKEY & PIERCE, P.L.C., P.O.BOX 8910, RESTON, VA, 20195, US

(21) Appl. No.: 11/139,694

(22) Filed: May 31, 2005

(30) Foreign Application Priority Data
Jun. 1, 2004 (DE) 10 2004 026 617.4

(51) Int. Cl. A61B 17/08

(52) U.S. Cl. 606/221

(57) ABSTRACT

Tissue can be clamped via an endorobot in a simple manner, and at the same time with a high degree of safety, by using a device which is received by the endorobot and which includes a clip, a drive unit and a control unit, as part of the endorobot for example. The clip is able to be advanced out of and retracted into a guide part of the endorobot independently by use of the drive unit, and of the control unit connected to the latter, without complicated mechanical connections to the outside.

The present application hereby claims priority under 35 U.S.C. §119 on German patent application number DE 10 2004 026 617.4 filed Jun. 1, 2004, the entire contents of which is hereby incorporated herein by reference.

### FIELD

The invention generally relates to a device for clamping tissue, preferably via an endorobot which can be brought into a treatment position in a hollow organ of a human or animal.

### BACKGROUND

US 2002/0049497 A1 discloses an endoscope which, for treatment of internal hemorrhages or for closure of open incision wounds inside the body of a living being, is equipped with a clip known per se. At one end, the clip has a forceps which is mounted in a guide part assigned to the endoscope. To clamp an internal hemorrhage or to close an incision wound, the clip is driven out from the guide part of the endoscope. Further, after activation of the clip, the tissue gripped by the forceps is clamped and the hemorrhage stopped or the incision wound closed.

Moreover, to ensure that less strain is placed on the patient in a diagnostic procedure performed by endoscopy, US 2004/0030454 A1 discloses an endoscope without cords or wires. This involves a capsule which is equipped with an imaging unit and with a transmitter for transmitting the recorded image data. A capsule of this kind, also called an endorobot, is introduced into the gastrointestinal tract preferably by the oral or anal route.

In hollow organs or hollow spaces of the body that are closed off from outside, the endorobot can be inserted through a small incision opening. To ensure that the endorobot does not move out of its examination position during a peristaltic contraction movement of the intestinal tract, a movement control unit is provided in the aforementioned known case. The movement control unit includes wings which are arranged around the circumference of the endorobot and can be deployed in order to control the movement of the endorobot. To secure the endorobot, the wings have mutually displaceable plates with which the tissue of the internal wall can be clamped.

### SUMMARY

An object of an embodiment of the present invention is directed to a device which permits clamping of tissue via an endorobot in a simplified manner and with guaranteed safety.

The advantageous simplicity of clamping tissue by the simple advance and retraction of a clip for an endorobot, dispensing with an endoscope and with the latter's complicated mechanics for control of the clip, is made possible by at least one embodiment of the inventive combination of the endorobot, which advances and retracts the clip, and of a drive unit in conjunction with a control unit, so that the tissue can be securely clamped by advancing and retracting the clip, without a fixed connection to the outside.

Multiple use of the device for clamping tissue in at least one embodiment may advantageously be made possible by use of a fixed connection of the clip to the endorobot, in which case the endorobot is released again from its treatment position by the clamped tissue dying and by associated detachment of the remaining tissue, so that, following one treatment, the device can be used again for further treatment, without any great outlay in maintenance terms.

The controlled release of the endorobot from its treatment position in at least one embodiment, even before the clamped tissue dies, can be advantageously ensured by the fact that the clip is connected to the endorobot via a releasable connection element.

In at least one embodiment, the clip can be released from the endorobot in a particularly user-friendly manner by remote control.

To aid or even ensure enhanced or even optimal success of treatment, the clip can expediently also be released via the releasable connection element by sensor control, for example depending on a temperature, or when a chemical reaction of the tissue starts.

The clip can advantageously be released in an effective and controlled manner by a separating unit assigned to the endorobot.

In an example embodiment of the invention, a releasable connection element is provided in the form of a predetermined break point which, when a retraction force imparted by the retraction of the clip is exceeded, can be released without separate control.

For diagnosis of diseased tissue, the clip is advantageously provided for cutting off the clamped tissue in the treatment position in the sense of collecting a tissue specimen, so that the collected tissue is available after a use of the endorobot for further investigations.

When it reaches its treatment position, the endorobot can be secured against inadvertent displacement by the fact that the clip is provided as an anchoring device for the endorobot.

Furthermore, in an example embodiment, the clip is also provided as a therapeutic device for clamping open incision wounds or for clamping hemorrhages inside the body of a living being.

### BRIEF DESCRIPTION OF THE DRAWINGS

The invention and further advantageous embodiments of the invention are explained in more detail below on the basis of illustrative example embodiments shown diagrammatically in the drawings, in which:

FIG. 1 shows an endorobot and a device, according to an embodiment of the invention, received by the latter for clamping tissue, with the components clip, drive unit and control unit each in a stand-by position,

FIG. 2 shows the endorobot according to FIG. 1, with the clip in a forward position in which it engages around the tissue,

FIG. 3 shows the endorobot according to FIG. 1, with the clip in a withdrawal position in which it clamps the tissue,

FIG. 4 shows the endorobot according to FIG. 1, supplemented by a first releasable connection element, in the form of a predetermined break point, for releasing the clip from the endorobot,

FIG. 5 shows the endorobot according to FIG. 1, with a second releasable connection element and a separating unit for releasing the clip from the endorobot,

FIG. 6 shows the endorobot according to FIG. 1, with the clip released from the endorobot and acting as therapeutic device, and

FIG. 7 shows the endorobot according to FIG. 1, with the clip fixedly connected to the endorobot and acting as a means for collecting a tissue specimen.

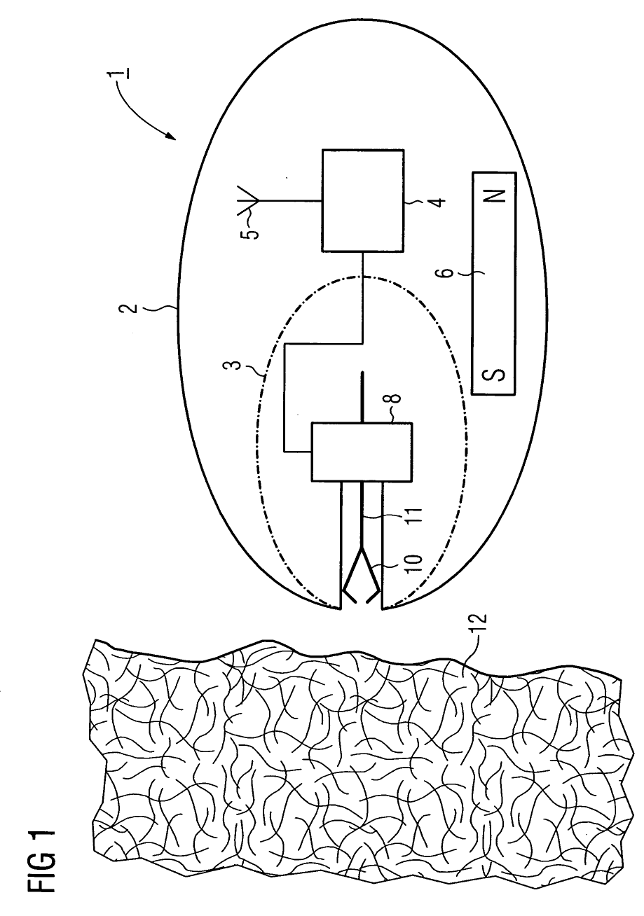

In FIG. 1, an endorobot 1 according to the invention with a device 3 for clamping tissue 12 is shown in a stand-by position.

### DETAILED DESCRIPTION OF THE EXAMPLE EMBODIMENTS

The endorobot 1, in an embodiment, has the form of a capsule that can be swallowed; its envelope 2 is made of a biocompatible material resistant to the digestive secretions arising in the gastrointestinal tract. A magnet element 6 assigned to the endorobot 1 permits contact-free navigation by means of a 3D gradient field generated in an examination area. A device 3 according to an embodiment of the invention, and described in more detail below, for clamping tissue 12 is arranged inside the capsule envelope 2 near one of its ends.

The device 3 according to an embodiment of the invention for clamping tissue 12 has three main components, namely a clip 11, a drive unit 8, and a control unit 4. The control unit 4 is used to control the drive unit 8 and may be connected by a wireless communication 5 to an external operating unit (not shown here). By use of a suitable control signal from the control unit 4, advance and retraction forces can be triggered which can be transmitted to the clip 11, mounted in a guide part, via a fixed connection to the drive unit 8 and to the endorobot 1. At one end, the clip 11 has a forceps 10 which is held in the guide part, is under tension and opens automatically outside the guide part.

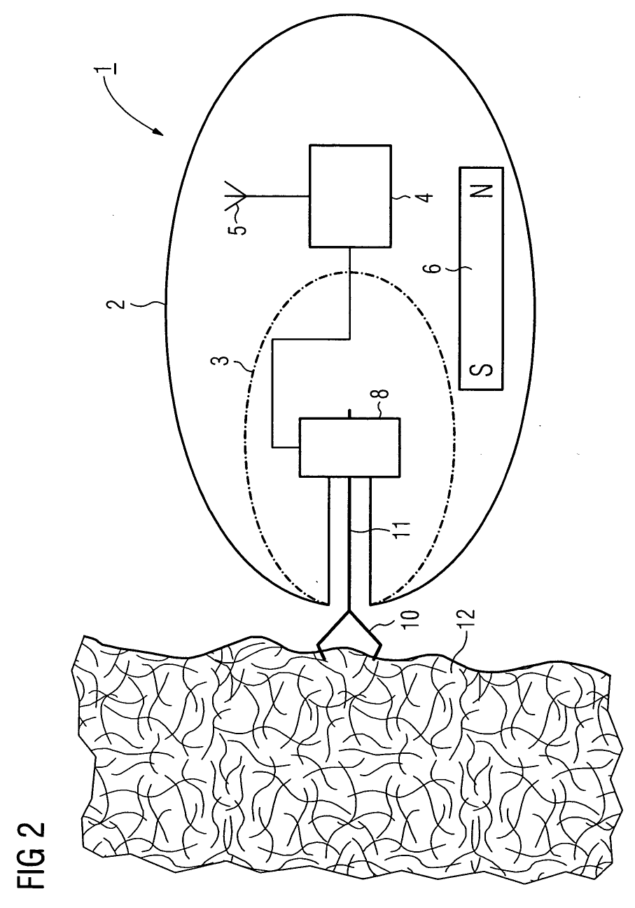

To clamp tissue 12 by use of an endorobot 1 at a previously defined location inside the body of a living being, a suitable control signal can be transmitted to the control unit 4 via wireless communication 5 in a treatment position. The control unit 4 activates the drive unit 8 so that an advance force that can be generated by the drive unit 8 can be triggered. The effect of the advance force acting on the clip 11 is, as shown in FIG. 2, that the clip 11 is pushed out of its guide part into a forward position, so that the forceps 10 held under tension engages around the tissue 12 lying in front of the guide part of the endorobot 1.

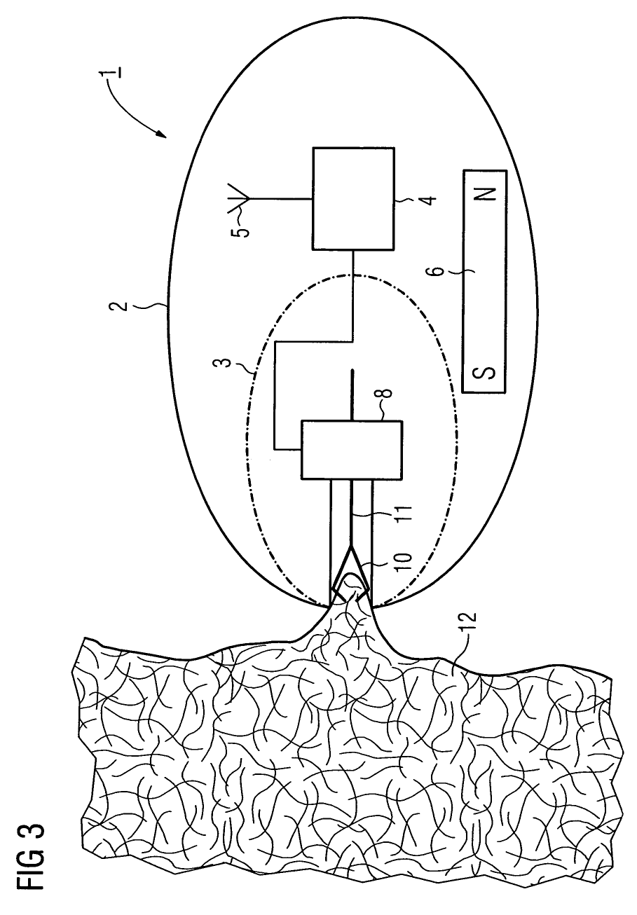

To clamp the tissue 12, a retraction force can be generated via the drive unit 8, for example after a predefined time or when a control signal is transmitted to the control unit 4 via the wireless communication 5. The effect of the retraction force is, as shown in FIG. 3, that the clip 11, together with the tissue 12 enclosed by the forceps 10, can be retracted into a withdrawal position in the guide part of the endorobot 1. By retraction of the clip 11, the forceps 10 open in the forward position is closed again, so that the tissue 12 enclosed between the forceps 10 can be clamped.

By clamping tissue 12 in a previously defined treatment position, the endorobot 1 can advantageously be securely anchored so that it is protected against unwanted movements, for example caused by peristaltic contractions of the intestinal tract.

The anchoring of the endorobot 1 can be released after a certain time, for example through death of the clamped tissue 12, so that the endorobot 1, in the case of an examination of the intestine, can be eliminated again by the natural route and without surgical intervention. The fixed connection between the clip 11 and the endorobot 1 permits expedient multiple use of the device 3 for clamping tissue 12, without the need for any great outlay for its maintenance. The device 3 can, for example, be prepared for further use simply by suitable disinfection of the components.

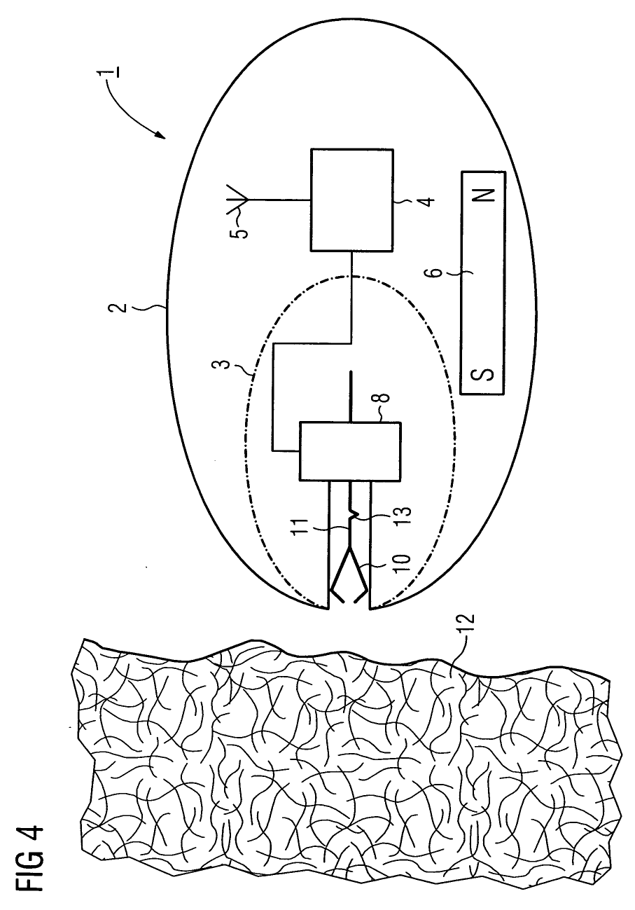

FIG. 4 shows the endorobot 1 according to an embodiment of the invention from FIG. 1 advantageously with an additional first releasable connection element 13 with which the clip 11 can be separated from the endorobot 1. The first releasable connection element 13 in this example is configured as a predetermined break point which breaks when a retraction force imparted by the retraction of the clip 11 after clamping of the tissue 12 is exceeded, such that the clip 11 can be separated from the endorobot 1 advantageously without external influence.

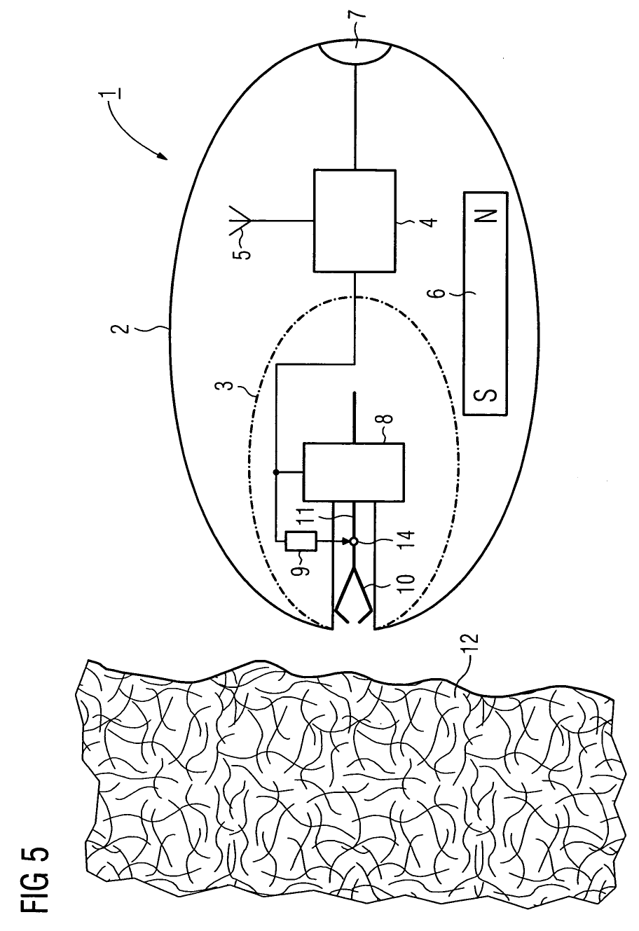

In a further advantageous embodiment, FIG. 5 shows the endorobot 1 with a second releasable connection element 14 which can be released in a controllable manner via a separating unit 9 connected to the control unit 4. To separate the clip 11 from the endorobot 1, a suitable control signal can be transmitted to the control unit 4 via wireless communication 5 in the treatment position. This control unit 4 activates the separating unit 9 so that the second releasable connection element 14 can be separated by use of a wedge assigned to the separating unit 9 being driven forward.

In an advantageous embodiment of the invention, in order to achieve optimally successful treatment, the second releasable connection element 14 can also be released by sensor control, namely by use of a sensor 7 connected to the control unit 4. To separate the clip 11 from the endorobot 1, the sensor 7 can transmit to the control unit 4 a corresponding control signal for activation of the separating unit 9 when a predefined sensor threshold value is reached. In this way, for example, a temperature sensor and/or a pressure sensor can be used which transmit to the control unit 4 a control signal for separating the clip 11 when predetermined temperature limit values and/or pressure limit values are reached. In principle, any desired sensors adapted for successful treatment can be used for sensor-controlled separation of the clip 11.

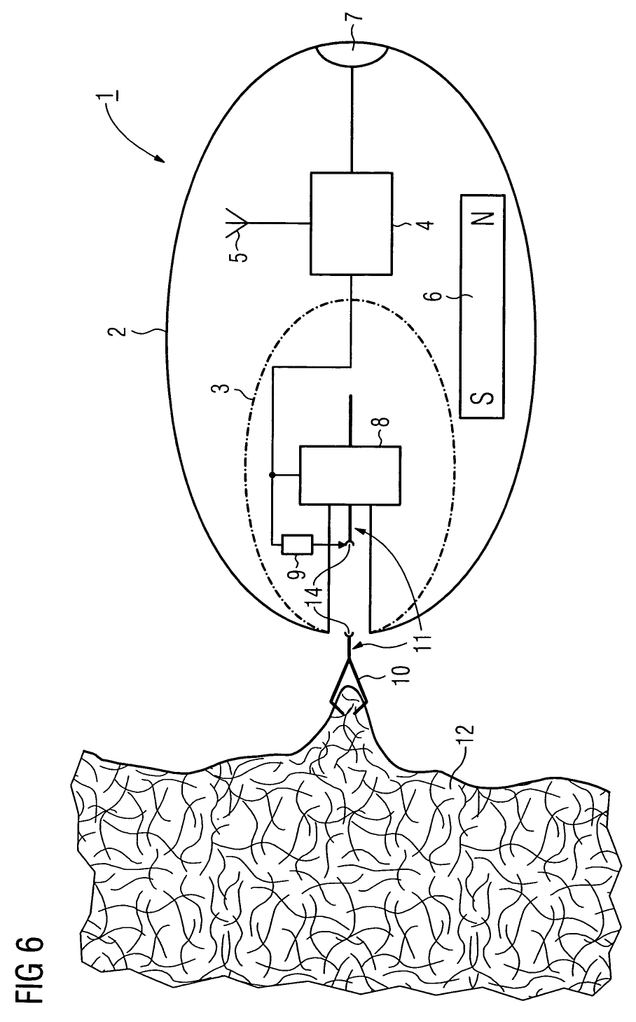

FIG. 6 shows the endorobot 1 from FIG. 5, the second releasable connection element 14 being released, and the clip 11, which clamps the tissue, remaining in the tissue 12, and the endorobot 1 being able to be navigated free from its treatment position. In the case of intestinal treatment, the endorobot 1 can in this way be eliminated by the natural route and does not necessarily have to be removed from the inside of the body of a living being by a surgical intervention. By contrast, the clip 11 remains in the treatment position until the tissue located between the forceps 10 of the clip 11 dies and separates from the rest of the tissue 12. The clip 11 separated in this way can then be eliminated by the natural route, like the endorobot 1.

In a further advantageous embodiment of the invention, the clip 11 can be used as a therapeutic device, in which case bleeding can be stopped by clamping the tissue, as may be the case in FIG. 6 for example. In addition, it is also conceivable for open incision wounds to be closed via a clip 11 clamping the tissue adjacent to the incision wound.

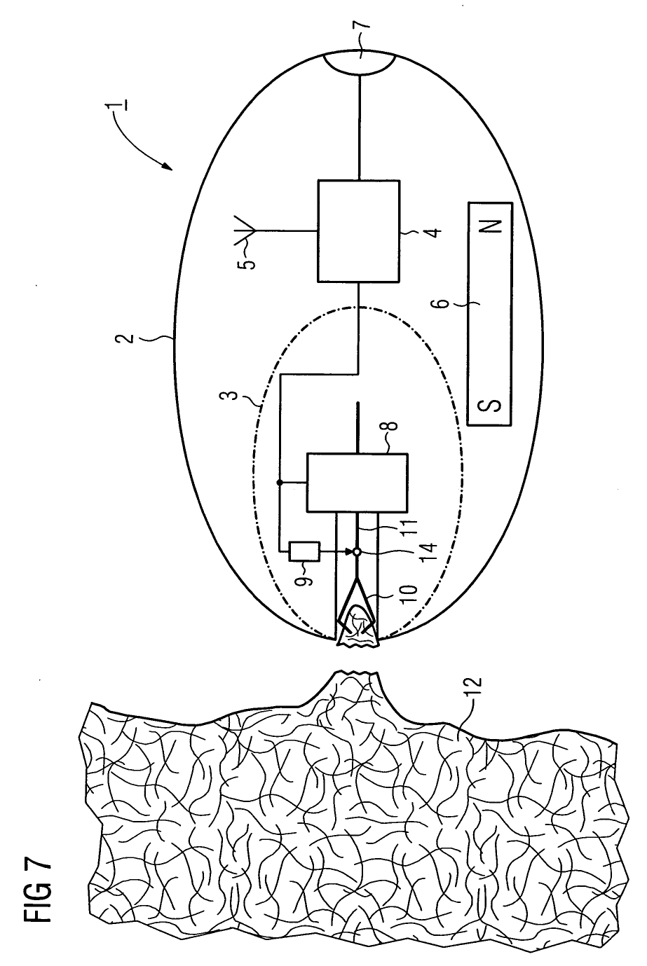

In a further advantageous embodiment, FIG. 7 shows the endorobot from FIG. 5, the clip 11 being provided for cutting off the clamped tissue in the sense of collecting a tissue specimen. FIG. 5 shows the clip 11 already in the withdrawal position in which a tissue specimen is located between the forceps 10 of the clip 11. The forceps 10 of the clip 11 is expediently designed in such a way that the jaws assigned to the forceps 10 close completely in the guide part of the endorobot 1 so as to cut off the tissue in the withdrawal position.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A device for clamping tissue via an endorobot which can be brought into a treatment position in a hollow organ of a human or animal, comprising:
a clip, mounted in a guide part and advanceable into a forward position, the clip being engageable around the tissue and retractable into a withdrawal position;
a drive unit, with which the clip is advanceable into the forward position and retractable into the withdrawal position; and
a control unit, with which the drive unit for advancing and retracting the clip is controllable.

2. The device as claimed in claim 1, wherein the clip is fixedly connected to the endorobot.

3. The device as claimed in claim 1, wherein the clip is releasably connected to the endorobot via a releasable connection element.

4. The device as claimed in claim 3, wherein the releasable connection element is releasable by remote control.

5. The device as claimed in claim 3, wherein the releasable connection element is releasable by sensor control.

6. The device as claimed in claim 3, wherein the releasable connection element is releasable via a separating unit.

7. The device as claimed in claim 3, wherein the releasable connection element is designed in the form of a predetermined break point.

8. The device as claimed in claim 2, wherein the clip is provided for cutting off the clamped tissue in the sense of collecting a tissue specimen.

9. The device as claimed in claim 1, wherein the clip is provided as an anchoring device, for anchoring the endorobot in the treatment position.

10. The device as claimed in claim 1, wherein the clip is provided as a therapeutic device.

11. The device as claimed in claim 4, wherein the releasable connection element is releasable via a separating unit.

12. The device as claimed in claim 5, wherein the releasable connection element is releasable via a separating unit.

13. An anchoring device, for anchoring an endorobot in a hollow organ of a human or animal, comprising:
a clip, engageable around the tissue and retractable into a withdrawal position;
a controller, for controlling advancement of the clip into the forward position and retraction of the clip into the withdrawal position.

14. The anchoring device of claim 13, further comprising:
a driver, for advancing the clip into the forward position and for retracting the clip into the withdrawal position, under control of the controller.

15. The anchoring device as claimed in claim 13, wherein the clip is fixedly connected to the endorobot.

16. The anchoring device as claimed in claim 13, wherein the clip is releasably connected to the endorobot via a releasable connection element.

17. An endorobot including the anchoring device of claim 13.

18. A device for clamping tissue via an endorobot which can be brought into a treatment position in a hollow organ of a human or animal, comprising:
a clip, mounted in a guide part and advanceable into a forward position, the clip being engageable around the tissue and retractable into a withdrawal position;
drive means for advancing the clip into the forward position and for retracting the clip into the withdrawal position; and
control means for controlling the drive menas.

19. The device as claimed in claim 18, wherein the clip is fixedly connected to the endorobot.

20. The device as claimed in claim 18, wherein the clip is releasably connected to the endorobot via a releasable connection element.
